(19) United States
(12) Patent Application Publication
(10) Pub. No.: US 2010/0035247 A1
(43) Pub. Date: Feb. 11, 2010

(54) Heterogeneous Assay of Analytes in Solution Using Polymers

(75) Inventor: Randall E. Burton, Billerica, MA (US)

Correspondence Address: WOLF GREENFIELD & SACKS, P.C., 600 ATLANTIC AVENUE, BOSTON, MA, 02210-2206, US

(73) Assignee: U.S. Genomics, Inc., Woburn, MA (US)

(21) Appl. No.: 12/084,542

(22) PCT Filed: Nov. 6, 2006
(86) PCT No.: PCT/US06/43139

§ 371 Date: Sep. 29, 2009

Related U.S. Application Data
(60) Provisional application No. 60/733,589, filed on Nov. 4, 2005.

(51) Int. Cl. C12Q 1/68; G01N 33/543; G01N 33/53

(52) U.S. Cl. 435/6; 436/518; 435/7.2; 435/7.1

(57) ABSTRACT

The invention relates to methods and systems for identifying, quantitating and/or analyzing analytes from samples. The analytes may be organic or inorganic in nature and include but are not limited to pathogens such as viruses.

### RELATED APPLICATIONS

This application claims priority to U.S. Provisional Application having Ser. No. 60/733,589, and entitled “HETEROGENEOUS ASSAY OF ANALYTES IN SOLUTION USING POLYMERS”, filed on Nov. 4, 2005, the entire contents of which are incorporated by reference herein.

### FIELD OF THE INVENTION

The invention relates, inter alia, to detection and quantitation of analytes from samples.

### BACKGROUND OF THE INVENTION

Multiplexing refers to the ability to analyze (e.g., detect) more than one, and preferably many, different substances simultaneously. The ability to perform a multiplexed analysis would be advantageous to a number of applications such as proteomics, clinical analysis of body fluids, biodefense, and the like. Applications involving a limited amount of sample or a low concentration of the substances to be detected particularly benefit from multiplexing capability. To be useful, multiplexing systems should demonstrate a high sensitivity, a wide dynamic range, and significant multiplexing capability.

There exists a need for a system that provides fast analysis of multiple analytes without compromising sensitivity, dynamic range and multiplexing capacity.

### SUMMARY OF THE INVENTION

The invention relates generally to analysis of analytes within samples using polymer based methods and compositions. The invention is capable of detecting, quantifying and also harvesting and further analyzing analytes in a sample. The methods and compositions relate to the use of polymers as unique identifiers for analytes of interest. A high degree of multiplexing is possible given the diversity in available polymers.

Thus, in one aspect, the invention provides a method for detecting an analyte in a sample comprising contacting a sample with a primary binding partner that is bound to a solid support thereby allowing an analyte present in the sample to bind to the primary binding partner, contacting the bound analyte with a secondary analyte-specific binding partner that is conjugated to a polymer, and analyzing the polymer that is indirectly bound to the analyte.

In one embodiment, the method comprises analyzing the polymer bound to the analyte comprises determining a labeling pattern of the polymer, wherein the labeling pattern of the polymer indicates the identity of the analyte.

In another aspect, the invention provides a method for detecting an analyte in a sample comprising contacting a sample with a primary analyte-specific binding partner and a secondary analyte-specific binding partner and detecting binding of an analyte to the primary and secondary analyte-specific binding partners, wherein the secondary analyte-specific binding partner is itself bound to a unique polymer. The method further comprises determining the identity of the polymer (e.g., by determining a labeling pattern of the polymer), wherein the identity of the polymer indicates the identity of the analyte.

In one embodiment, the analyte is a plurality of analytes, the primary binding partner is a plurality of primary binding partners, and the secondary analyte-specific binding partner is a plurality of secondary analyte-specific binding partners.

The analyte may be a nucleic acid, a carbohydrate, a protein, a peptide, a lipid, a toxin, a cell, a spore, a cellular fragment or a spore fragment but it is not so limited. The analyte however is normally different from the polymer attached to the secondary analyte-specific binding partner, particularly if the polymer is labeled with probes after binding to the analyte.

In one embodiment, the primary binding partner is a primary analyte-specific binding partner. The primary binding partner may be a nucleic acid or a peptide or protein, but it is not so limited. In one embodiment, the binding partner is an antibody or an antigen-binding antibody fragment.

The polymer may be a nucleic acid, such as a DNA or RNA. It may be naturally occurring or non-naturally occurring. The polymer may be elongated prior to and/or simultaneously with its analysis.

The secondary analyte-specific binding partner may be a nucleic acid or a peptide or protein, but it is not so limited. In one embodiment, the secondary analyte-specific binding partner is an antibody or an antigen-binding antibody fragment. The secondary analyte-specific binding partner may be identical to the primary binding partner.

In one embodiment, the secondary analyte-specific binding partner is conjugated to a detectable label. In another embodiment, the primary binding partner and the secondary analyte-specific binding partner is each labeled with a member of a FRET pair.

In one embodiment, the labeling pattern of the polymer is a binding pattern of one or more sequence-specific probes to the polymer. The one or more sequence-specific probes may be conjugated to detectable labels. In one embodiment, the labeling pattern of the polymer is a pattern of detectable labels incorporated into the polymer. In another embodiment, the labeling pattern of the polymer is a binding pattern of one or more restriction endonucleases to the polymer. The labeling pattern may alternatively be a unique detectable label incorporated into the polymer or a probe bound thereto or conjugated to a probe.

In one embodiment, the labeling pattern of the polymer is determined using a focused flow through an electric field.

In one embodiment, the method further comprises analyzing the analyte bound to the secondary analyte-specific binding partner.

In another aspect, the invention provides a composition comprising a nucleic acid bound to an antibody or an antigen-binding antibody fragment and having a unique label, wherein the unique label is comprised of one or more incorporated detectable labels, one or more bound detectable sequence-specific nucleic acid probes, or one or more bound detectable proteins.

In yet another aspect, the invention provides a composition comprising a nucleic acid bound to an antibody or an antigen-binding antibody fragment, wherein the nucleic acid is 10-1000 kilobases in length.

In some embodiments, the nucleic acid is DNA such as but not limited to synthetic DNA.

In another embodiment, the nucleic acid is bound to the Fc region of the antibody or antigen-binding antibody fragment.

Many of the embodiments recited above apply equally to all aspects of the invention as would be apparent to one of ordinary skill in the art. These and other aspects and embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and/or the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

FIG. 1 illustrates a sample contacted with a set of primary analyte-specific binding partners such as antibodies. The antibodies are bound to a solid support such as a bead or a plastic surface.

FIG. 2 illustrates the binding of one analyte to one primary antibody, while the remaining sample and its analyte contents are washed away.

FIG. 3 illustrates the binding of a secondary analyte-specific binding partner such as antibody to its respective analyte. The secondary antibody is conjugated to a polymer such as a DNA. The remaining unbound secondary antibodies are washed away. Note that each secondary antibody with its own specific binding affinity (as denoted by different numbers) is bound to a uniquely labeled DNA (as denoted by the number and pattern of small circles).

FIG. 4 illustrates the binding of one analyte and one secondary antibody with a uniquely labeled polymer conjugated thereto. The environment can then be changed to dissociate the analyte/antibody complexes such that the secondary antibody is released from the solid support. In some embodiments, the secondary antibody is also released from the analyte, although the invention is not so limited.

FIG. 5 illustrates the conjugation of a secondary antibody (anti-GST antibody) to a DNA (lambda DNA) using a short LNA attached to the Fc portion of the antibody. In the Figure, lambda DNA is provided with 5′ overhangs and the antibody is attached to the 3′ end of a locked nucleic acid having sequence complementary to the lambda 5′ overhangs.

The Figures are illustrative only and are not required for enablement of the invention.

### DETAILED DESCRIPTION OF THE INVENTION

The invention provides in its broadest sense a system for detecting one or more analytes from a sample. The invention employs analyte-specific binding partners that are conjugated to polymers. Each analyte to be detected has a corresponding polymer which is identified by a unique barcode. The unique barcode of the polymer indicates the analyte-specificity of the binding partner to which it is bound and thus the identity of the analyte.

The method is particularly suited to determining analyte content in a sample wherein the sample is rare or the analyte concentration is low. The invention allows more than one and preferably several different analytes to be detected simultaneously, thereby conserving sample. In other words, the method is capable of a high degree of multiplexing. The degree of multiplexing will depend on the particular application and the number of analytes to be detected. For example, the degree of multiplexing may be 2 (i.e., 2 analytes can be detected in a single analysis), 3, 4, 5, 6, 7, 8, 9, 10, at least 20, at least 50, at least 100, at least 500, or higher.

The degree of multiplexing also appears to be limited by the throughput rate of the specific detection/interrogation system used. The detection/interrogation system may be a single molecule detection system, and more particularly a single molecule linear detection system, such as the GeneEngine™ system.

The polymer as used herein is any molecule capable of being elongated, conjugated to binding partners, and uniquely labeled. A polymer that is elongated is a polymer that is stretched. It may be partially or completely stretched, provided that it is capable of being analyzed in a linear manner (i.e., without any two regions of the polymer overlapping with one and other). A polymer this is uniquely labeled is a polymer that is labeled in a way that is different and distinguishable from the manner is which all other polymers being analyzed are labeled.

Thus, the polymer may be nucleic acid, amino acid, carbohydrate or lipid in nature, but it is not so limited. In an important embodiment, the polymer is a nucleic acid, whether naturally occurring or not. The nucleic acid may be naturally or non-naturally occurring DNA or RNA, such as genomic DNA, mitochondrial DNA, mRNA, cDNA, mRNA, miRNA, PNA or LNA, or a combination thereof, as described herein. Non-naturally occurring polymers such as bacterial artificial chromosomes (BACs) and yeast artificial chromosomes (YACs) can also be used. Preferably, a non-naturally occurring polymer (e.g., synthesized DNA) is used in order to control mapping and conjugation to analyte-specific binding partners. Harvest and isolation of nucleic acids are routinely performed in the art and suitable methods can be found in standard molecular biology textbooks. (See, for example, Maniatis' Handbook of Molecular Biology.)

It is important that the polymer be uniquely labeled since this label is used to identify a particular analyte or class of analytes. This unique labeling pattern of the polymer is referred to herein as the barcode. The labeling pattern or barcode is one or more detectable (and in some instances unique) labels present on or in a polymer, which uniquely identify that polymer (as well as others identical to it that bind an identical analyte via an analyte-specific binding partner). Each labeling pattern or barcode is associated with one analyte or one analyte class. The labeling pattern or barcode may be a spatial pattern of detectable labels incorporated into the polymer during its synthesis. Alternatively, it may be a binding pattern of one or more sequence-specific or structure-specific probes. The probes are detectable either intrinsically or via conjugation to detectable labels. The labeling pattern may also be a combination of the foregoing labeling strategies.

If determining the barcode requires contacting the polymer with probes, such probes may be added prior to, simultaneously with, or following the addition of the polymer to a sample. That is, the polymer may be labeled before, during or after binding of the analyte-specific binding partners to their respective analytes, provided that the conditions for binding of any of these pairs does not disrupt any of the other binding interactions.

As an example, the polymer may be a nucleic acid that is labeled with bisPNA (as discussed in greater detail herein). BisPNA binding to nucleic acids such as DNAs is stable and can withstand the change in environment required to disrupt the immunocomplexes in the assay. Accordingly, the DNA can be labeled with bisPNA probes prior to performing the assay. As another example, the polymer may be nucleic acid that is labeled with proteins such as restriction endonucleases (preferably that are modified to bind but not cleave nucleic acids or are present in conditions that accomplish the same result such as for example reduced divalent cation conditions). The binding of such proteins to nucleic acids such as DNA may not withstand the change in conditions require to disrupt the immunocomplexes and as a result, the polymer may be labeled after it is released from the solid support.

The polymer may be of any length, provided that it comprises sufficient sequence information to be unique and in some instances to allow for a greater degree of multiplexing.

As shown in Example 2, the polymer may be about 50 kilobases. Alternatively, the polymer may be less than that (e.g., 10-20 kilobases) or greater than that (e.g., 150 kilobases).

Depending on the embodiment, the polymer may be at least 1, at least 2, at least 5, at least 10, at least 15, at least 20, at least 25, at least 30, at least 40, at least 50, at least 75, at least 100, at least 150, at least 200, at least 300, at least 400, at least 500, at least 600, at least 700, at least 800, at least 900, at least 1000 kilobases or more (including every integer there between as if each was explicitly recited herein).

In one embodiment, the polymer is conjugated to the binding partner via a cleavable bond (as discussed below). Whether in this embodiment or others, the invention also contemplates the release of the polymer from the binding partner prior to polymer analysis using polymer analysis systems as described herein. The polymer may be cleaved using light, chemical or enzymatic means as will be known in the art.

The polymer acts as an surrogate marker for the analyte being detected. Each polymer is associated with a particular analyte binding specificity. It is therefore possible to determine the analyte binding capacity of a polymer by “reading” its barcode. Presence of an analyte is determined based on the presence of the unique polymer barcode. As will be discussed herein, it is possible that all secondary binding partners are conjugated to the same detectable label if such label is in addition to the polymer. This is because such a label merely indicates that the analyte is present with the polymer barcode indicating the exact identity of the analyte.

In some embodiments, it is preferred that the polymer be flexible. This is the case with nucleic acids such as DNA which normally exists as a random coil and is stretched only during its analysis. Stretching the DNA during such analysis enables a higher degree of multiplexing since each polymer can be distinguished based on the relative spatial location of probes or detectable labels. Stretching is not required however during probe or analyte-specific binding partner incubation.

The invention further contemplates analysis of polymers in a compact, non-elongated form. This can be useful if each polymer labeling pattern is uniquely detected irrespective of spatial location of probes or detectable labels. For example, it is possible that each analyte-specific polymer is labeled with a unique label and the presence of the label regardless of its position along the length of the polymer is used to identify the polymer (and consequently the analyte bound thereto). This approach will be best suited to applications that do not require extensive multiplexing. It should be understood that this approach will therefore not require a linear analysis system nor will it require elongation of the polymer prior to or during polymer analysis.

A binding partner as used herein is a compound that binds to an analyte with a desired level of specificity. Generally, the specificity is at a level at which the binding partner binds preferentially to the analyte of interest rather than other compounds or analytes. Its affinity for the analyte of interest may be at least 2-fold, at least 5-fold, at least 10-fold, or more than its affinity for another compound. Binding partners with the greatest differential affinity are preferred in most embodiments. The binding partners can be of any nature including but not limited to nucleic acid (e.g., aptamers), peptide, carbohydrate, lipid, and the like. A common form of binding partner is an antibody or an antigen-binding antibody fragment. Antibodies include IgG, IgA, IgM, IgE, IgD as well as antibody variants such as single chain antibodies. Suitable antibody fragments contain an antigen-binding site and thus include but are not limited to Fv, Fab and F(ab)₂ fragments. A nucleic acid based binding partner such as an oligonucleotide can be used to recognize and bind DNA or RNA based analytes. The nucleic acid based binding partner can be DNA, RNA, LNA or PNA, although it is not so limited. It can also be a combination of one or more of these elements and/or can comprise other nucleic acid mimics.

Binding partners can be primary or secondary. Primary binding partners are those bound to for example, a solid support such as a bead, a column, a plastic support, a well, a disk, an array, etc. and to which the analyte first binds. The primary binding partner usually is not conjugated to a polymer used to identify an analyte.

In some embodiments, the primary binding partners are not analyte-specific, although they may be epitope- or domain-specific. This may be the case if every analyte of interest contains a common marker or epitope which can be used to bind the analyte to the solid support. For example, if the assay is interested only in phosphorylated analytes (e.g., tyrosine phosphorylated proteins or peptides), then it is possible to use a primary binding partner that is specific for phosphorylated tyrosine residues. This may also be the case if the analytes contained in a sample are commonly modified to possess a marker (e.g., an epitope) to which the primary binding partner binds. For example, the analytes may all be phosphorylated or methylated and the primary binding partners would be phosphorylation or methylation specific.

Secondary binding partners are those that bind to an analyte that is already bound to the primary binding partner. Secondary binding partners are conjugated to polymers that are used to identify an analyte. Preferably, the primary and secondary binding partners bind to separate regions on an analyte regardless of whether those regions are identical in terms of sequence or structure. In other words, binding of either the primary or secondary binding partners should not effectively compete with or interfere with the binding of the other to the analyte. Where applicable, the secondary analyte-specific binding partners may be detectably labeled. In such instances, a labeled binding partner has multiple labels (but preferably not polymers) conjugated thereto in order in increase signal.

It is to be understood that in some embodiments the analyte is itself detectable.

An analyte as used herein is a molecule or compound being detected, quantitated or analyzed according to the invention. Analytes can be any molecule for which a binding partner is available. In its broadest sense, the analytes can be detected using virtually any molecular recognition system, such as but not limited to antibodies, aptamers, carbohydrates, etc. The analytes can be organic or inorganic in nature, and in important embodiments, they include proteins, peptides, toxins such as microbial toxins, nucleic acids such as oligonucleotides, pathogens such as bacteria, viruses, fungi, parasites, mycobacteria, and the like. Although the analytes to be detected are not size restricted, those that are equal to or less than 500 nm are preferred in some embodiments.

The invention can be applied to the detection and optionally identification and/or quantification of any analyte, but most preferably rare analytes which would otherwise be costly to detect. One example of one such analyte is a biohazardous or biowarfare agent. These agents can be biological or chemical in nature. Biological biowarfare agents can be classified broadly as pathogens (including spores thereof) or toxins. As used herein, a pathogen (including a spore thereof) is an agent capable of entering a subject such as a human and infecting that subject. Examples of pathogens include infectious agents such bacteria, viruses, fungi, parasites, mycobacteria and the like. Prions may also be considered pathogens to the extent they are thought to be the transmitting agent for CJD and like diseases. As used herein, a toxin is a pathogen-derived agent that causes disease and often death in a subject without also causing an infection. It derives from pathogens and so may be harvested from such pathogens. Alternatively, it may be synthesized apart from pathogen sources. Biologicals may be weaponized (i.e., aerosolized) for maximum spread. Examples of biowarfare agents include those listed and categorized by the CDC.

CDC Category A agents include Bacillus anthracis (otherwise known as anthrax), Clostridium botulinum and its toxin (causative agent for botulism), Yersinia pestis (causative agent for the plague), variola major (causative agent for small pox), Francisella tularensis (causative agent for tularemia), and viral hemorrhagic fever causing agents such as filoviruses Ebola and Marburg and arenaviruses such as Lassa, Machupo and Junin.

CDC Category B agents include Brucellosis (Brucella species), epsilon toxin of Clostridium perfringens, food safety threats such as Salmonella species, E. coli and Shigella, Glanders (Burkholderia mallei), Melioidosis (Burkholderia pseudomallei), Psittacosis (Chlamydia psittaci), Q fever (Coxiella bumetii), ricin toxin (from Ricinus communis—castor beans), Staphylococcal enterotoxin B, Typhus fever (Rickettsia prowazekii), viral encephalitis (alphaviruses, e.g., Venezuelan equine encephalitis, eastern equine encephalitis, western equine encephalitis), and water safety threats such as e.g., Vibrio cholerae, Cryptosporidium parvum.

CDC Category C agents include emerging infectious diseases such as Nipah virus and hantavirus.

Other pathogens that can be detected using the methods of the invention include N. gonorrhea, H. pylori, Staphylococcus spp., Streptococcus spp. such as Streptococcus pneumoniae, Syphilis; viruses such as SARS virus, Hepatitis A, B and C viruses, Herpes virus, HIV, West Nile virus, Influenza A virus, poliovirus, rhinovirus; and parasites such as Giardia.

Examples of toxins include abrin, ricin and strychnine. Further examples of toxins include toxins produced by Corynebacterium diphtheriae (diphtheria), Bordetella pertussis (whooping cough), Vibrio cholerae (cholera), Bacillus anthracis (anthrax), Clostridium botulinum (botulism), Clostridium tetani (tetanus), and enterohemorrhagic Escherichia coli (bloody diarrhea and hemolytic uremic syndrome), Staphylococcus aureus alpha toxin, Shiga toxin (ST), cytotoxic necrotizing factor type 1 (CNF 1), E. coli heat-stable toxin (ST), botulinum, tetanus neurotoxins, S. aureus toxic shock syndrome toxin (TSST), Aeromonas hydrophila aerolysin, Clostridium perfringens perfringolysin O, E. coli hemolysin, Listeria monocytogenes listeriolysin O, Streptococcus pneumoniae pneumolysin, Streptococcus pyogenes streptolysine O, Pseudomonas aeruginosa exotoxin A, E. coli DNF, E. coli LT, E. coli CLDT, E. coli EAST, Bacillus anthracis edema factor, Bordetella pertussis dermonecrotic toxin, Clostridium botulinum C2 toxin, C. botulinum C3 toxin, Clostridium difficile toxin A, and C. difficile toxin B.

Examples of chemicals that can be detected include arsenic, arsine, benzene, blister agents/vesicants, blood agents, bromine, borombenzylcyanide, chlorine, choking/lung/pulmonary agents, cyanide, distilled mustard, fentanyls and other opioids, mercury, mustard gas, nerve agents, nitrogen mustard, organic solvents, paraquat, phosgene, phosphine, sarin, sesqui mustard, stibine, sulfur mustard, warfarin, tabun, and the like.

The foregoing lists of infections are not intended to be exhaustive but rather exemplary.

The number of detectable analytes is usually not limited by number of different signals (or labels) but more often by the number of resolvable sites in the polymer.

The methods can detect a plurality of analytes using a plurality of different analyte-specific binding partners and a plurality of polymers. A plurality as used herein is more than one and can be at least 2, at least 3, at least 4, at least 5, at least 10, at least 25, at least 50, at least 75, at least 100, at least 200, at least 500, or more.

The sample to be tested for analyte presence and/or amount can be derived from virtually any source and will depend primarily on the analyte being detected. The sample may be a biological sample from a subject such as a bodily fluid or tissue. The term tissue as used herein refers to both localized and disseminated cell populations including, but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea and lung. Biological fluids include saliva, sperm, serum, plasma, blood, lymph and urine, but are not so limited. Both invasive and non-invasive techniques can be used to obtain such samples and these are known to those of ordinary skill in the art.

Alternatively, the sample may be an environmental sample such as an air sample or a water sample. In this latter embodiment, the sample may be checked for, for example, chemical or biological warfare agents such as those recited herein. If the sample is an air sample, it will generally require dissolution in a liquid base such as a buffered solution. This is usually also the case with solid samples.

The analyte being detected can dictate whether the sample needs to be further manipulated prior to analysis. In some embodiments, it may be necessary to disrupt larger analytes such as pathogens prior to contact with the binding partner. Disruption can be mechanical, including acoustic disruption (e.g., ultrasound based disruption), and may be carried out to varying degrees. For example, a sample may be disrupted to the point of rupturing cell walls and/or cell membranes and releasing cell wall fragments, intracellular organelles, proteins, lipids, and/or genomic DNA, all of which may be analytes.

Depending on the expected concentration of the analyte being detected, the sample may be diluted or concentrated prior to analysis. Dilution will generally involve mixing of the sample with a larger volume of solution. Concentration can be accomplished in a number of ways known in the art including but not limited to centrifugation, filtering, and the like. Concentration may also be accomplished using flow directed concentration methods.

The invention can be used to determine the concentration or absolute amount of an analyte in a sample. The concentration or amount of the analyte is determined by measuring the amount of signal from a polymer that is or was bound to an analyte (via the binding partner). The number of analyte-specific binding partners must be greater than the number of analytes in the sample in order to provide meaningful quantitative data. If the analyte concentration in the test solution is very high, the test solution can be diluted in order to quantitate analyte concentration accurately. The signal level can be compared to a standard calibration curve that is prepared prior to or at the same time as the test solution is analyzed.

The standard calibration curve is a plot of signal intensity (y-axis) as a function of analyte concentration (x-axis). Those of ordinary skill will be familiar with the generation of such curves.

The various embodiments of the invention described herein make specific reference to a polymer that is DNA and primary and second analyte-specific binding partners that are antibodies. It is to be understood however that these descriptions are intended as illustrative only and are not meant to limit the scope of the invention. Thus, any polymer can be used in the methods of the invention. Similarly, any analyte-specific binding partner can be used as either or both the first and second analyte-specific binding partners.

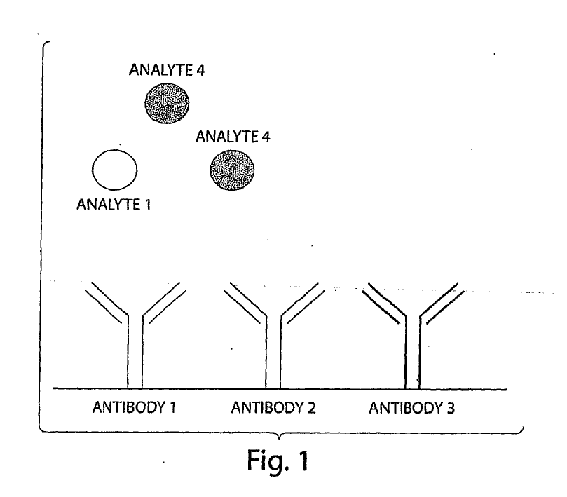

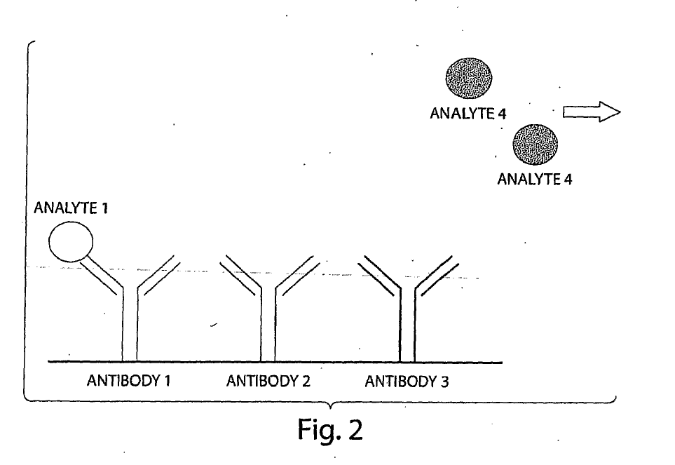

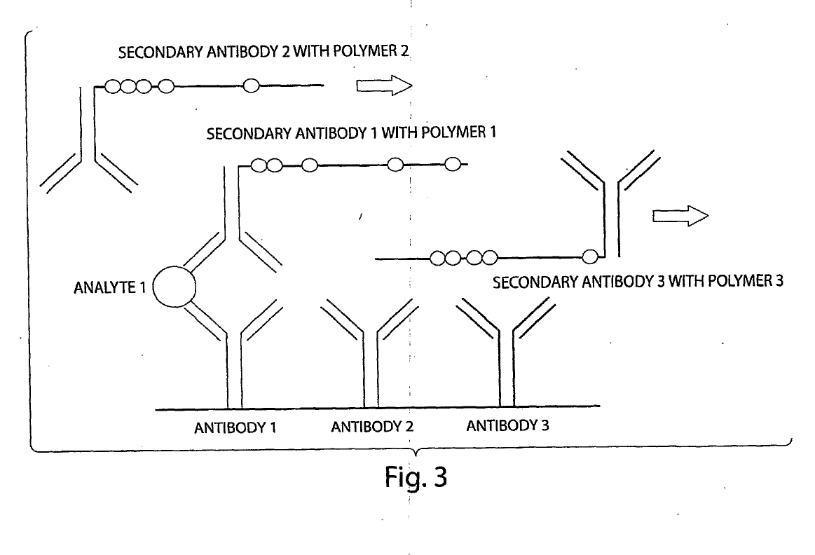

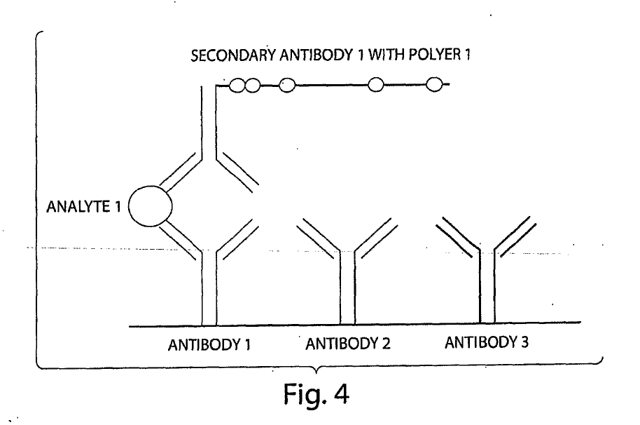

Thus, in one illustrative embodiment, one or a plurality of primary binding partners, preferably bound to a solid support, are contacted with a sample which may contain the analyte of interest. If it does, the analyte binds to its respective binding partner and the remainder of the sample is removed (e.g., washed away). The secondary binding partner is then added. The secondary binding partner has conjugated to it a polymer which is specific for the particular analyte to which the binding partner binds. The secondary binding partner binds to its respective analyte, if present, and the unbound secondary binding partners are removed (e.g., washed away). The conditions can then be changed in order to release the secondary binding partner from the solid support (e.g., either with or without the analyte bound thereto). The released secondary binding partner and its respective attached polymer are then analyzed. In some embodiments, the polymer is released from the binding partner. These steps are illustrated in FIGS. 1-4.

The polymer is preferably a DNA having a particular sequence unique to that DNA and referred to herein as the barcode. The binding partners are preferably antibodies or antigen-binding antibody fragments thereof. The incubation time and conditions are dictated by the particular analyte and the binding affinity of the antibody. One of ordinary skill in the art is capable of determining these parameters. The type of detection system to be used or available will dictate the type of detectable labels that are bound to or incorporated into the polymer and/or binding partner. The presence of an analyte in the sample is indicated by the presence of the particular DNA barcode. The identity of the analyte is dictated by the sequence (or barcode) of the DNA.

The DNA can be labeled prior to, during or following analyte binding. It can be labeled using labeled nucleotides that are incorporated during its synthesis, or by binding to it one or more labeled probes, although as discussed herein such labeling is not limited in this regard. The DNA barcode may be comprised of one or more spatially separated labels.

In another illustrative example, a polymer such as a DNA is bound to a detectable probe such as a fluorescent sequence-specific probe that recognizes a particular sequence motif in the DNA and binds to it. The molecule is interrogated and the location of binding of the probe is determined. Each DNA may have one or more bound probes. The binding pattern of the probes along the DNA can be used to identify the DNA and consequently its analyte binding specificity.

Preferably, DNAs having different sequences (and thus different barcodes) have antibodies with different binding specificities bound thereto.

A sample containing different analytes may be incubated with primary analyte-specific antibodies. The sample may also be incubated with secondary antibodies, or alternatively, such antibodies may be added later in time. The secondary antibodies are conjugated to polymers. During the incubation period, the secondary analyte-specific binding partner binds to the analyte either simultaneously with or after binding of the analyte to the primary binding partner.

The sample and polymer may be analyzed using a single polymer analysis system which in some instances is also a linear polymer analysis system such as but not limited to GeneEngine™. When placed in a moving fluid, DNA is stretched in the microfluidic chip and translocated into an interrogation zone (e.g., a spot of excitation light). In some embodiments, the spot diameter is about 0.5 μm, and therefore much smaller than the stretched DNA length which is about 34 μm for 100 kb DNA.

In one embodiment, the GeneEngine™ platform is used with focusing flow design. This arrangement provides interrogation of all polymers, improves polymer stretching, and moves the sample through the center of the excitation beam for more efficient detection. This arrangement therefore increases signal to noise (S/N) ratio and minimizes dispersion of excitation power.

Using such flow configurations, it is also possible to concentrate and/or redirect polymers of interest, such as polymers having an analyte of interest bound thereto (via the binding partner). In a flow system, this is easily accomplished by redirecting flow into a collection vessel. The collected polymer can then be manipulated, possibly to dissociate the analyte from its respective binding partner(s). The analyte whether in free or bound form can then be analyzed in greater detail. For example, if the analyte is a nucleic acid, it may be analyzed via PCR.

Individual sequence sites on the polymer that may contribute to a barcode should not be located so close to each other as to not be detectable as separate sites (i.e., the distance between these sites should be greater than the minimal resolution distance). Reference can be made to published U.S. Patent Application Publication No. 2003-0059822 A1 and/or published PCT Application No. WO 03/025540 for a discussion of minimal resolution distances. Additionally, the length of each site contributing to a barcode should not exceed the resolution limit of the detection system. For example, if the interrogation is performed at a 1 kilobase resolution (e.g., the resolution limit for a given analysis), then the length of each site preferably does not exceed 0.34 μm (i.e., the length of 1 kb B-form DNA [2]). The length of the sequence site may be defined for example as the length of a given nucleotide sequence bound by a probe.

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The organic bases include adenine, uracil, guanine, thymine, cytosine and inosine. The nucleic acids may be single or double stranded. Nucleic acids can be obtained from natural sources, or can be synthesized using a nucleic acid synthesizer.

As used herein with respect to linked units of a polymer including a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting for example the individual units of a particular nucleic acid, are most common. Natural linkages include, for instance, amide, ester and thioester linkages. The individual units of a nucleic acid analyzed by the methods of the invention may be linked, however, by synthetic or modified linkages. Nucleic acids where the units are linked by covalent bonds will be most common but those that include hydrogen bonded units are also embraced by the invention. It is to be understood that all possibilities regarding nucleic acids apply equally to nucleic acid targets and nucleic acid probes.

In some embodiments, the invention embraces nucleic acid derivatives as polymers and/or probes. As used herein, a “nucleic acid derivative” is a non-naturally occurring nucleic acid or a unit thereof. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

The polymers and probes if comprising nucleic acid components can be stabilized in part by the use of backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, the polymer or probe is a nucleic acid that is a peptide nucleic acid (PNA), a bisPNA clamp, a pseudocomplementary PNA, a locked nucleic acid (LNA), DNA, RNA, or co-nucleic acids of the above such as DNA-LNA co-nucleic acids. In some instances, the nucleic acid target can also be comprised of any of these elements.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes.

PNAs are synthesized from monomers connected by a peptide bond (Nielsen, P. E. et al. Petide Nucleic Acids. Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

Several types of PNA designs exist, and these include single strand PNA (ssPNA), bisPNA and pseudocomplementary PNA (pcPNA).

The structure of PNA/DNA complex depends on the particular PNA and its sequence. Single stranded PNA (ssPNA) binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids. Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNAJPNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize with a Hoogsteen pairing. BisPNAs can differ in the positioning of the linker around the target DNA strands.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand.

Locked nucleic acids (LNA) are modified RNA nucleotides. (See, for example, Braasch and Corey, Chem. Biol., 2001, 8(1): 1-7.) LNAs form hybrids with DNA which are at least as stable as PNA/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs. Therefore, production of mixed LNA/DNA sequences is as simple as that of mixed PNA/peptide sequences.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine particularly useful if positive charges are desired in the PNA), and the like. Various PNA modifications are known and probes incorporating such modifications are commercially available from sources such as Boston Probes, Inc.

As stated herein, one way of generating a labeling pattern or a barcode is to use one or more sequence-specific probes. “Sequence-specific” when used in the context of a probe for a nucleic acid polymer means that the probe recognizes a particular linear (or quasi-linear) arrangement of nucleotides or derivatives thereof. In preferred embodiments, the probe is itself composed of nucleic acid elements such as DNA, RNA, PNA and LNA elements and combinations thereof (as discussed below). In preferred embodiments, the linear arrangement includes contiguous nucleotides or derivatives thereof that each binds to a corresponding complementary nucleotide in the probe. In some embodiments, however, the sequence may not be contiguous as there may be one, two, or more nucleotides that do not have corresponding complementary residues on the probe.

Any molecule that is capable of recognizing a polymer such as a nucleic acid with structural or sequence specificity can be used as a sequence-specific probe. In most instances, such probes will form at least a Watson-Crick bond with the nucleic acid polymer. In other instances, the nucleic acid probe can form a Hoogsteen bond with the nucleic acid polymer, thereby forming a triplex. A nucleic acid probe that binds by Hoogsteen binding enters the major groove of a nucleic acid polymer and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid polymer. BisPNA probes, for instance, are capable of both Watson-Crick and Hoogsteen binding to a nucleic acid.

The nucleic acid probes of the invention can be any length ranging from at least 4 nucleotides to in excess of 1000 nucleotides. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 15 nucleotides, at least 20 nucleotides, or at least 25 nucleotides, or more, in length. It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other.

The probes are preferably single stranded, but they are not so limited. For example, when the probe is a bisPNA it can adopt a secondary structure with the nucleic acid polymer resulting in a triple helix conformation, with one region of the bisPNA clamp forming Hoogsteen bonds with the backbone of the polymer and another region of the bisPNA clamp forming Watson-Crick bonds with the nucleotide bases of the polymer.

The nucleic acid probe hybridizes to a complementary sequence within the nucleic acid polymer. The specificity of binding can be manipulated based on the hybridization conditions. For example, salt concentration and temperature can be modulated in order to vary the range of sequences recognized by the nucleic acid probes. Those of ordinary skill in the art will be able to determine optimum conditions for a desired specificity.

As stated herein, the polymer may be directly labeled. As an example, if the polymer is a nucleic acid, it may be labeled through the use of sequence-specific probes that bind to the polymer in a sequence-specific manner. The sequence-specific probes are labeled with a detectable label (e.g., a fluorophore or a radioisotope).

The nucleic acid however can also be synthesized in a manner that incorporates fluorophores directly into the growing nucleic acid. For example, it is possible to synthesize nucleic acids de novo (e.g., using automated nucleic acid synthesizers) using fluorescently labeled nucleotides. Such nucleotides are commercially available from suppliers such as Amersham Pharmacia Biotech, Molecular Probes, and New England Nuclear/Perkin Elmer.

It is also possible to label nucleic acids by the introduction of active amino or thiol groups during synthesis of the nucleic acids. (Proudnikov and Mirabekov, Nucleic Acid Research, 24:4535-4532, 1996.) An extensive description of modification procedures that can be performed on a nucleic acid polymer can be found in Hermanson, G. T., Bioconjugate Techniques, Academic Press, Inc., San Diego, 1996, which is incorporated by reference herein. One of the methods is based on the introduction of aldehyde groups by partial depurination of DNA. Fluorescent labels with an attached hydrazine group are efficiently coupled with the aldehyde groups and the hydrazine bonds are stabilized by reduction with sodium labeling efficiencies around 60%. The reaction of cytosine with bisulfite in the presence of an excess of an amine fluorophore leads to transamination at the N4 position (Hermanson, 1996). Reaction conditions such as pH, amine fluorophore concentration, and incubation time and temperature affect the yield of products formed. At high concentrations of the amine fluorophore (3M), transamination can approach 100% (Draper and Gold, 1980).

Probes or analyte-specific binding partner may also be labeled, for example, using a detectable label. A detectable label is a moiety, the presence of which can be ascertained directly or indirectly. Generally, detection of the label involves the creation of a detectable signal such as for example an emission of energy. The label may be of a chemical, peptide or nucleic acid nature although it is not so limited. The nature of label used will depend on a variety of factors, including the nature of the analysis being conducted, the type of the energy source and detector used and the type of polymer, analyte, probe and primary and secondary analyte-specific binding partners. The label should be sterically and chemically compatible with the constituents to which it is bound.

The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.). Generally the detectable label can be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl)aminonaphthalene-1-sulfonic acid (EDANS), 4-acetamido-4′-isothiocyanatostilbene-2, 2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′, 6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl)aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., P³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle (such as quantum dots described for example in U.S. Pat. No. 6,207,392 and commercially available from Quantum Dot Corporation and Evident Technologies), a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, p-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like. Antibody fragments include Fab, F(ab)₂, Fd and antibody fragments which include a CDR3 region.

In some embodiments, primary and secondary analyte-specific binding partners are conjugated with donor and acceptor fluorophores, respectively, that form a FRET (fluorescence resonance energy transfer) pair. In this case, a blue laser light is used to excite fluorescence of donor fluorophores. A portion of the energy absorbed by the donors can be transferred to acceptor fluorophores if they are spatially close enough to the donor molecules (i.e., the distance between them must approximate or be less than the Forster radius or the energy transfer radius). Once the acceptor fluorophore absorbs the energy, it in turn fluoresces in its characteristic emission wavelength. Since energy transfer is possible only when the acceptor and donor are located in close proximity, acceptor fluorescence is unlikely if the secondary analyte-specific binding partner is not bound to the analyte which is in turn bound to the primary analyte-specific binding partner. Acceptor fluorescence therefore can be used to determine presence and optionally concentration of analyte.

FRET can be used, for example, in an array format in order to determine if a particular secondary antibody is bound regardless of the identity of the analyte to which it binds. Alternatively, the secondary binding partner may be labeled detectably without labeling of the primary binding partner.

Labeling of the secondary binding partner is also useful for establishing the orientation of the polymer attached thereto. For example, during polymer analysis, the orientation of the polymer is not always apparent. If the polymer is bound at one end by a binding partner that is itself detectably labeled, then its orientation will be known. This results in more usable information and more rapid analysis.

FRET alone generally requires only one excitation source (and thus wavelength) and usually only one detector. The detector may be set to either the emission spectrum of the donor or acceptor fluorophore. It is set to the donor fluorophore emission spectrum if FRET is detected by quenching of donor fluorescence. Alternatively, it is set to the acceptor fluorophore emission spectrum if FRET is detected by acceptor fluorophore emission. In some embodiments, FRET emissions of both donor and acceptor fluorophores can be detected. In still other embodiments, the donor is excited with polarized light and polarization of both emission spectra is detected.

FRET requires the use of a FRET fluorophore pair. FRET fluorophore pairs are two fluorophores that are capable of undergoing FRET to produce or eliminate a detectable signal when positioned in proximity to one another. Examples of donors include Alexa 488, Alexa 546, BODIPY 493, Oyster 556, Fluor (FAM), Cy3 and TMR (Tamra). Examples of acceptors include Cy5, Alexa 594, Alexa 647 and Oyster 656. Cy5 can work as a donor with Cy3, TMR or Alexa 546, as an example. FRET should be possible with any fluorophore pair having fluorescence maxima spaced at 50-100 nm from each other.

The polymer may be labeled in a sequence non-specific manner in addition to the barcode labeling discussed herein. For example, if the polymer is a nucleic acid such as DNA, then its backbone may be stained with a backbone label. Examples of backbone stains that label nucleic acids in a sequence non-specific manner include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

In instances in which the nucleic acid polymer is stained with a non-specific backbone stain, the detection system should be capable of detecting and distinguishing between three distinct signals (i.e., one for the backbone, one for the sequence-specific sites that make up the barcode or labeling pattern, and one for the analyte or the secondary analyte-specific binding partner). Such a system should then be equipped for three color detection and three color excitation. If the FRET configuration is used as described herein, then the number of excitation lasers and/or detectors may be reduced.

As an example, in one embodiment, three different lasers are used for excitation at the following wavelengths: 488 nm (blue), 532 nm (green), and 633 nm (red). These lasers excite fluorescence of Alexa 488, TMR (tetramethylrhodamine), and TOTO-3 fluorophores, respectively. Fluorescence from all these fluorophores can be detected independently. As an example of fluorescence strategy, the sequence-specific probes or the DNA itself may be labeled with Alexa 488 fluorophores, the secondary antibodies may be labeled with TMR, and the DNA backbone may be labeled with TOTO-3. TOTO-3 is an intercalating dye that non-specifically stains DNA in a length-proportional manner. In this configuration, Alexa 488 fluorescence is used to determine the barcode or labeling pattern, TMR fluorescence bound to the DNA is indicative of analyte presence in the test solution (and thus bound to the DNA), and TOTO-3 fluorescence provides context for the barcode signal by labeling part of or the entire length of the DNA polymer, in some instances thereby allowing fine tuning of the barcode. TMR fluorescence can also be used to quantitate analyte concentration in the solution, as discussed herein. Another suitable set of fluorophores that can be used is the combination of POPO-1, TMR and Alexa 647 (or Cy5) which are excited by 442, 532 and 633 nm lasers respectively.

As used herein, “conjugated” means two entities stably bound to one another by any physicochemical means. It is important that the nature of the attachment is such that it does not substantially impair the effectiveness of either entity. Keeping these parameters in mind, any covalent or non-covalent linkage known to those of ordinary skill in the art is contemplated unless explicitly stated otherwise herein. Such means and methods of attachment are known to those of ordinary skill in the art. Conjugation can be performed using standard techniques common to those of ordinary skill in the art. For example, U.S. Pat. Nos. 3,940,475 and 3,645,090 demonstrate conjugation of fluorophores and enzymes to antibodies.

The various components described herein can be conjugated to each other by any mechanism known in the art. For instance, functional groups which are reactive with various labels include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

The secondary analyte-specific binding partners can be conjugated to the polymer and the detectable labels can be conjugated to all suitable components of the system by covalent or non-covalent means, whether directly or indirectly. Linkers and/or spacers may be used in some instances.

Linkers can be any of a variety of molecules, preferably nonactive, such as nucleotides or multiple nucleotides, straight or even branched saturated or unsaturated carbon chains of C₁-C₃₀, phospholipids, amino acids, and in particular glycine, and the like, whether naturally occurring or synthetic. Additional linkers include alkyl and alkenyl carbonates, carbamates, and carbamides. These are all related and may add polar functionality to the linkers such as the C₁-C₃₀ previously mentioned. As used herein, the terms linker and spacer are used interchangeably.

A wide variety of spacers can be used, many of which are commercially available, for example, from sources such as Boston Probes, Inc. (now Applied Biosystems). Spacers are not limited to organic spacers, and rather can be inorganic also (e.g., —O—Si—O—, or O—P—O—). Additionally, they can be heterogeneous in nature (e.g., composed of organic and inorganic elements). Essentially, any molecule having the appropriate size restrictions and capable of being linked to the various components such as fluorophore and probe can be used as a linker. Examples include the E linker (which also functions as a solubility enhancer), the X linker which is similar to the E linker, the O linker which is a glycol linker, and the P linker which includes a primary aromatic amino group (all supplied by Boston Probes, Inc., now Applied Biosystems). Other suitable linkers are acetyl linkers, 4-aminobenzoic acid containing linkers, Fmoc linkers, 4-aminobenzoic acid linkers, 8-amino-3,6-dioxactanoic acid linkers, succinimidyl maleimidyl methyl cyclohexane carboxylate linkers, succinyl linkers, and the like. Another example of a suitable linker is that described by Haralambidis et al. in U.S. Pat. No. 5,525,465, issued on Jun. 11, 1996.

The length of the spacer can vary depending upon the application and the nature of the components being conjugated (e.g., the polymer and the primary analyte-specific binding partner and the distance that can be tolerated between target sites on a polymer).

The conjugations or modifications described herein employ routine chemistry, which is known to those skilled in the art of chemistry. The use of linkers such as mono- and hetero-bifunctional linkers is documented in the literature (e.g., Herman-Son, 1996) and will not be repeated here.

The linker molecules may be homo-bifunctional or hetero-bifunctional cross-linkers, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl)suberate, bis[2-(succinimidooxycarbonyloxy)ethyl]sulfone, disuccinimidyl suberate, disuccinimidyl tartarate, dimethyl adipimate•2HCl, dimethyl pimelimidate•2HCl, dimethyl suberimidate-2 HCl, and ethylene glycolbis-[succinimidyl-[succinate]]. Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido) butyl]-3′-[2′-pyridyldithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide•2HCl, 4-(4-N-maleimidophenyl)-butyric acid hydrazide•2HCl, and 3-[2-pyridyldithio]propionyl hydrazide. The cross-linkers are bis-[β-4-azidosalicylamido)ethyl]disulfide and glutaraldehyde.

Amine or thiol groups may be added at any nucleotide of a synthetic nucleic acid so as to provide a point of attachment for a bifunctional cross-linker molecule. The nucleic acid may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

Non-covalent methods of conjugation may also be used to bind the binding partner to the polymer, or to bind a detectable label to a probe, a polymer or an analyte-specific binding partner, for example. Non-covalent conjugation includes hydrophobic interactions, ionic interactions, high affinity interactions such as biotin-avidin and biotin-streptavidin complexation and other affinity interactions. As an example, a molecule such as avidin may be attached the nucleic acid, and its binding partner biotin may be attached to the analyte-specific antibody.

In some instances, it may be desirable to use a linker or spacer comprising a bond that is cleavable under certain conditions. For example, the bond can be one that cleaves under normal physiological conditions or that can be caused to cleave specifically upon application of a stimulus such as light, whereby the primary analyte-specific binding partner is released leaving the polymer intact. Readily cleavable bonds include readily hydrolyzable bonds, for example, ester bonds, amide bonds and Schiffs base-type bonds. Bonds which are cleavable by light are known in the art.

The polymers may be analyzed using a single molecule analysis system (e.g., a single polymer analysis system). A single molecule detection system is capable of analyzing single molecules separately from other molecules. Such a system may be capable of analyzing single molecules either in a linear manner and/or in their totality. In certain embodiments in which detection is based predominately on the presence or absence of a signal, linear analysis may not be required. However, there are other embodiments embraced by the invention which would benefit from the ability to linearly analyze molecules (preferably nucleic acids) in a sample. These include applications in which the sequence of the nucleic acid is desired, or in which the polymers are distinguished based on spatial labeling pattern (e.g., a barcode) rather than a unique detectable label.

Thus, the polymers can be analyzed using linear polymer analysis systems. A linear polymer analysis system is a system that analyzes polymers such as nucleic acids, in a linear manner (i.e., starting at one location on the polymer and then proceeding linearly in either direction therefrom). The polymers being analyzed are generally intact and do not require cleavage in order to be analyzed by the single or linear polymer analysis systems envisioned by the invention. These systems and processes do not degrade the polymers in order to analyze them.

As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time) that can then be translated into a map, with knowledge of the velocity of the polymer. It is to be understood that in some embodiments, the polymer is attached to a solid support, while in others it is free flowing. In either case, the velocity of the polymer as it moves past, for example, an interaction station or a detector, will aid in determining the position of the labels relative to each other and relative to other detectable markers that may be present on the polymer.

An example of a suitable system is the GeneEngine™ (U.S. Genomics, Inc., Woburn, Mass.). The Gene Engine™ system is described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference herein in their entirety. This system is both a single molecule analysis system and a linear polymer analysis system. It allows, for example, single nucleic acids to be passed through an interaction station in a linear manner, whereby the nucleotides in the nucleic acid are interrogated individually in order to determine whether there is a detectable label conjugated to the nucleic acid. Interrogation involves exposing the nucleic acid to an energy source such as optical radiation of a set wavelength. The mechanism for signal emission and detection will depend on the type of label sought to be detected, as described herein.

This system comprises an optical source for emitting optical radiation; an interaction station for receiving the optical radiation and for receiving a polymer that is exposed to the optical radiation to produce detectable signals; and a processor constructed and arranged to analyze the polymer based on the detected radiation including the signals.

In one embodiment, the interaction station includes a localized radiation spot. In a further embodiment, the system further comprises a microchannel that is constructed to receive and advance the polymer through the localized radiation spot, and which optionally may produce the localized radiation spot. In another embodiment, the system further comprises a polarizer, wherein the optical source includes a laser constructed to emit a beam of radiation and the polarizer is arranged to polarize the beam. While laser beams are intrinsically polarized, certain diode lasers would benefit from the use of a polarizer. In some embodiments, the localized radiation spot is produced using a slit located in the interaction station. The slit may have a slit width in the range of 1 nm to 500 nm, or in the range of 10 nm to 100 nm. In some embodiments, the polarizer is arranged to polarize the beam prior to reaching the slit. In other embodiments, the polarizer is arranged to polarize the beam in parallel to the width of the slit.

In yet another embodiment, the optical source is a light source integrated on a chip. Excitation light may also be delivered using an external fiber or an integrated light guide. In the latter instance, the system would further comprise a secondary light source from an external laser that is delivered to the chip.

The analysis may also comprise generating optical radiation of a known wavelength to produce a localized radiation spot; passing a polymer through a microchannel; irradiating the polymer at the localized radiation spot; sequentially detecting radiation resulting from interaction of the polymer with the optical radiation at the localized radiation spot; and analyzing the polymer based on the detected radiation.

In one embodiment, the method further employs an electric field to pass the polymer through the microchannel. In another embodiment, detecting includes collecting the signals over time while the polymer is passing through the microchannel.

The systems described herein will encompass at least one detection system. The nature of such detection systems will depend upon the nature of the detectable label. The detection system can be selected from any number of detection systems known in the art. These include an electron spin resonance (ESR) detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

Other nucleic acid analytical methods can be used in the methods of the invention. These include fiber-fluorescence in situ hybridization (fiber-FISH) (Bensimon, A. et al., Science 265(5181):2096-2098 (1997)). In fiber-FISH, nucleic acid molecules are elongated and fixed on a surface by molecular combing. Hybridization with fluorescently labeled probe sequences allows determination of sequence landmarks on the nucleic acid molecules. The method requires fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other polymer analysis systems can also be used, and the invention is not intended to be limited to solely those listed herein.

Optical detectable signals are generated, detected and stored in a database. The signals can be analyzed to determine structural information about the nucleic acid. The signals can be analyzed by assessing the intensity of the signal to determine structural information about the nucleic acid. The computer may be the same computer used to collect data about the nucleic acids, or may be a separate computer dedicated to data analysis. A suitable computer system to implement embodiments of the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection-mechanism. Computer programs for data analysis of the detected signals are readily available from CCD (charge coupled device) manufacturers.

The present invention is further illustrated by the following Examples, which in no way should be construed as further limiting. The entire contents of all of the references (including literature references, issued patents, published patent applications, and co-pending patent applications) cited throughout this application are expressly incorporated by reference herein.

### EXAMPLES

### Example 1

The invention provides an assay that employs one set of capture units fixed to a solid support, such as a magnetic bead or a plastic surface (e.g., the bottom of a 96-well plate). In one embodiment, these capture units specifically recognize and bind analytes. This binding is probed using a second set of capture units that bind to an identical or a different region of the analyte. An identical region as used in this context means a duplicated region. Any secondary capture units that bind to the analyte are then fixed to the solid support, while the remainder are washed away. Bound capture units are then released from the surface using more aggressive wash conditions (e.g., lower pH for antibodies, higher temperature for oligonucleotides, etc.). The second set of capture units may contain a detectable label such as a fluorescent dye. If a non-polymer label on the capture unit is used to identify and/or measure the analyte, then the degree of multiplexing may be more limited than the method provided herein. In the present assay, the secondary capture units are attached to polymers, such as synthetic DNA molecules, designed to have distinctive barcodes. The barcodes on the DNA molecules will be used to identify the capture units, and the quantity of the analyte specific to these capture units will be determined from the number of DNA events displaying that barcode.

### Example 2

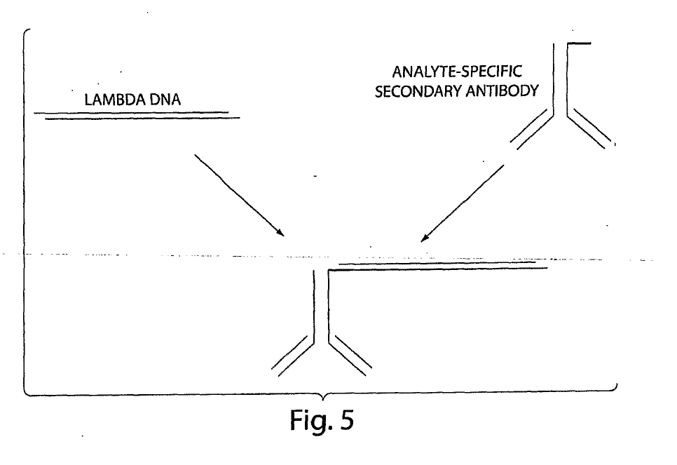

The Example shown in FIG. 5 illustrates the conjugation of lambda DNA to an antibody. Lambda DNA is on the order of about 50 kilobases and inherently contains 5′ overhangs. The antibody is synthesized or first conjugated to a short nucleic acid (in this case, an LNA) having a sequence complementary to one 5′ overhang on the lambda DNA. The antibody is an anti-GST antibody. The antibody is attached to the 3′ end of the LNA.

Conjugation of polymers such as nucleic acids to binding partners such as antibodies (or fragments thereof) can be accomplished as known in the art. (See for example Zhou et al., Nucleic Acids Research 21(25):6038-6039, 1993; Adler, Adv Clin Chem. 39:239-92, 2005.)

### EQUIVALENTS

It should be understood that the preceding is merely a detailed description of certain embodiments. It therefore should be apparent to those of ordinary skill in the art that various modifications and equivalents can be made without departing from the spirit and scope of the invention, and with no more than routine experimentation.

All references, patents and patent applications that are recited in this application are incorporated by reference herein in their entirety.

What is claimed is:

1. A method for detecting an analyte in a sample comprising
contacting a sample with a primary binding partner that is bound to a solid support thereby allowing an analyte present in the sample to bind to the primary binding partner,
contacting the bound analyte with a secondary analyte-specific binding partner that is conjugated to a polymer, and
analyzing the polymer bound to the analyte,
wherein the polymer indicates presence of the analyte.

2. The method of claim 1, wherein analyzing the polymer bound to the analyte comprises determining a labeling pattern of the polymer, wherein the labeling pattern of the polymer indicates the identity of the analyte.

3. The method of claim 1, wherein the analyte is a plurality of analytes, the primary binding partner is a plurality of primary binding partners, and the secondary analyte-specific binding partner is a plurality of secondary analyte-specific binding partners.

4. The method of claim 1, wherein the primary binding partner is a primary analyte-specific binding partner.

5. The method of claim 1, wherein the polymer is a nucleic acid.

6. The method of claim 1, wherein the primary binding partner is an antibody or an antigen-binding antibody fragment.

7. The method of claim 1, wherein the secondary analyte-specific binding partner is an antibody or an antigen-binding antibody fragment.

8. The method of claim 1, wherein the secondary analyte-specific binding partner is conjugated to a detectable label.

9. The method of claim 1, wherein the primary binding partner and the secondary analyte-specific binding partner is each labeled with a member of a FRET pair.

10. The method of claim 2, wherein the labeling pattern of the polymer is a binding pattern of one or more sequence-specific probes to the polymer.

11. The method of claim 10, wherein the one or more sequence-specific probes are conjugated to detectable labels.

12. The method of claim 2, wherein the labeling pattern of the polymer is a pattern of detectable labels incorporated into the polymer.

13. The method of claim 2, wherein the labeling pattern of the polymer is a binding pattern of one or more restriction endonucleases to the polymer.

14. The method of claim 1, further comprising analyzing the analyte bound to the secondary analyte-specific binding partner.

15. The method of claim 1, wherein the analyte is a nucleic acid, a carbohydrate, a protein, a peptide, a lipid, a toxin, a cell, a spore, a cellular fragment or a spore fragment.

16. The method of claim 1, wherein the polymer is elongated prior to or simultaneously with its analysis.

17. The method of claim 2, wherein the labeling pattern of the polymer is determined using a focused flow through an electric field.

18. A composition comprising
a nucleic acid bound to an antibody or an antigen-binding antibody fragment and having a unique label,
wherein the unique label is comprised of one or more incorporated detectable labels, one or more bound detectable sequence-specific nucleic acid probes, or one or more bound detectable proteins.

19. A composition comprising
a nucleic acid bound to an antibody or an antigen-binding antibody fragment,
wherein the nucleic acid is 10-1000 kilobases in length.

20. The composition of claim 18, wherein the nucleic acid is DNA.

21. The composition of claim 20, wherein the DNA is synthetic DNA.

22. The composition of claim 18, wherein the nucleic acid is bound to the Fc region of the antibody or antigen-binding antibody fragment.
